(19) United States
(12) Patent Application Publication
(10) Pub. No.: US 2008/0003203 A1
(43) Pub. Date: Jan. 3, 2008

(54) Pseudotyped baculovirus to stimulate immunogenicity against avian influenza

(75) Inventors: Yu-Chen Hu, Hsinchu (TW); Ding-Gang Yang, Hsinchu (TW); Hung-Jen Liu, Hsinchu (TW)

Correspondence Address: JACKSON WALKER LLP, 901 MAIN STREET, SUITE 6000, DALLAS, TX, 75202-3797, US

(73) Assignee: National Tsing Hua University, Hsinchu (TW)

(21) Appl. No.: 11/594,610

(22) Filed: Nov. 8, 2006

Related U.S. Application Data
(60) Provisional application No. 60/816,889, filed on Jun. 28, 2006.

(51) Int. Cl. A61K 48/00; C12N 15/86

(52) U.S. Cl. 424/93.2; 435/456

(57) ABSTRACT

The current invention relates to vaccines that use baculovirus vectors to expose a host organism to an immunogen, thereby eliciting an immune response. A pseudo-typed baculovirus is used to display hemagglutinin on the cell membrane in order to increase host immunogenicity.

### RELATED APPLICATIONS

This application claims priority to U.S. Provisional Patent Application, Application No. 60/816,889, entitled “Pseudotyped Baculovirus as a Vaccine Against Avian Influenza” filed on Jun. 28, 2006, having Yu-Chen Hu, Ding-Gang Yang, and Hung-Jen Liu listed as the inventors, the entire content of which is hereby incorporated by reference.

### BACKGROUND

The current invention relates to the use of a pseudotyped baculovirus for stimulating immunogenicity against avian influenza in a host.

### Avian Influenza (AI)

Avian influenza (AI) viruses, type A members of the Orthomyxoviridae family, have eight negative sense RNA segments encoding 10 proteins (Alexander, 2000). Among these genes, the hemagglutinin (HA) and neuraminidase (NA) genes encode virulence-associated surface glycoproteins, and antibody to either, inhibits infection (Iwasaki, 2004) or prevents disease (Johansson, 1993). HA is the most abundant surface glycoprotein, is responsible for attachment of virus to terminal sialic acid residues on host cell receptors, and mediates fusion between viral and cellular membranes (Alexander, 2000). To date, viruses of 15 HA and 9 NA subtypes have been identified in avian species (Takada, 1999). Among the 15 HA subtypes, the highly pathogenic (HP) phenotypes are only associated with some strains of the H5 or H7 HA subtype (Lee, 2004).

AI virus has been an economic threat to commercial poultry industry worldwide. For example, the severe outbreak of the H5N1 strain of AI in many East Asia countries in 2003-2004 has led to the depopulation of more than one hundred million birds. Since 1997, AI virus infection has taken on new significance, with increasing numbers of cases involving bird-to-human transmission. Human infection with avian H5N1 virus was first reported in Hong Kong in 1997, which resulted in 18 people infected and 6 deaths (Mounts, 1999). Episodes caused by various AI virus strains (including H5 and H7 subtypes) have continued to re-appear in other countries such as Vietnam, Thailand, Cambodia, China, Indonesia and the Netherlands, etc. The expanding geographic distribution of AI virus (H5N1) infections, with recent outbreaks in Kazakstan, Mongolia, and Russia, indicates that more human populations are at risk (Liu, 2005). Among the various virus strains, H5N1 is the most highly pathogenic to humans and has caused 142 confirmed human cases and 74 deaths from 2003 to 2005 in Asian countries (http://www.who.int/csr/disease/avian_influenza/country/cases_table_{—}2006_{—}01_{—}05/en/index.html). In January 2006 (as of Jan. 23, 2006), H5N1 infection led to 1 more death in China and 4 more deaths in Turkey (http://www.who.int/csr/disease/avian_influenza/country/cases_table_{—}2006_{—}01_{—}19/en/index.html). The fatality rate among hospitalized patients has been high, ranging from 33% (Hong Kong), to 80% (Vietnam) and 100% in Cambodia (WHO, 2005). The widespread epidemic and high fatality rate highlight the urgent need to develop vaccines for the control of emerging H5N1 AI viruses and the prevention of infection. Currently, human vaccines against H5N1 virus are not available (WHO, 2005). Avian vaccine against H5N2 viruses of North American lineage has been used in Mexico since 1995 (Lee, 2004), but avian vaccines against H5N1 viruses are not available (WHO, 2005).

### Baculovirus

Baculovirus was originally regarded to infect solely insects and invertebrates and has been widely used for the production of numerous recombinant proteins in insect cells (Kost, 2002). In 1995, AcMNPV (Autographa californica multiple nuclear polyhedrosis virus) was found capable of transducing human hepatoma cells and expressing proteins under the control of mammalian promoters (Hofmann, 1995). Since then, subsequent studies have rapidly expanded the list of permissive cells that include cell lines originating from human, rodent, porcine and bovine species (for review, see Kost, 2002; Hu, 2005). Besides, baculovirus is capable of transducing nondividing cells (van Loo, 2001) and primary cells such as rat articular chondrocytes (Ho, 2004). These discoveries have led to the attempts to develop baculovirus vectors carrying mammalian expression cassettes for in vitro and in vivo gene therapy studies, drug screening, studies of HSV and CMV virology, studies of gene function, cell/cell fusion assay, recombinant protein production and viral vector production (for review, see Hu, 2005). Several baculovirus vectors are commercially available, and would be known by one of ordinary skill in the art (see Hu, 2005, the entire content of which is hereby incorporated by reference). Use of these vectors would be considered within the scope of the current invention.

Baculovirus infects insect cells and transduces mammalian cells first by attaching to the cellular receptor and internalizing by receptor-mediated endocytosis. The attachment is mediated by a glycoprotein gp64 (molecular mass≈64 kD) on the viral envelope (Monsma, 1996; Oomens, 1995). The gp64 gene encodes an N-terminal signal sequence and a mature domain that includes the transmembrane domain and cytoplasmic domain (CTD). After expression, gp64 is transported to the cell membrane directed by the signal peptide, where the mature domains oligomerize and are presented on the surface of infected cells as homotrimers (Jarvis, 1994). Upon budding of the newly synthesized nucleocapsid (budded form), the gp64 CTD interacts with nucleocapsids and directs the incorporation of gp64 into the budding virion and display on the viral envelope.

In 1995, the feature of gp64 was first exploited for the surface display of exogenous peptides (Boublik, 1995). The surface display can be performed by fusing a heterologous protein (or peptide) in-frame at the N-terminus of gp64 gene under the control of polyhedrin or p10 promoter. The use of several types of promoters for driving gene expression is known to those skilled in the art, and would be considered within the scope of this invention (see Hu, 2005, the entire content of which is hereby incorporated by reference). The fusion protein, after expression as an additional copy, is translocated to the plasma membrane and incorporated into the viral envelope upon virus budding. Similar strategy has been applied to construct avidin-displaying baculovirus, which shows a 5-fold increase in transduction efficiency (compared to the wild-type baculovirus) in rat glioma cells (Raty, 2004). Baculovirus displaying heterologous envelope proteins such as vesicular stomatitis virus G protein (VSVG) has also been constructed, which transduces human hepatoma and rat neuronal cells at efficiencies 10˜100-fold greater than baculovirus lacking VSVG (Barsoum, 1997). This pseudotyped virus also transduces cell lines that are transduced at very low levels by the unmodified baculovirus, thus broadening the tropism. In contrast, specific targeting of baculovirus to mammalian cells by displaying single-chain antibody fragment specific for the carcinoembryonic antigen (CEA) or synthetic IgG binding domains is also demonstrated (Mottershead, 2000).

Host responses to baculovirus uptake, either in vitro or in vivo, were not evaluated until Gronowski et al. reported that administration of live baculovirus in vitro induces interferon-α and β (IFNα/β) production from human and murine cell lines and induces in vivo protection of mice from encephalomyocarditis virus infection (Gronowski, 1999). Subsequently, Abe et al. found that intranasal inoculation with a wild-type baculovirus elicits a strong innate immune response that protects mice from a lethal challenge of influenza virus (Abe, 2003). Moreover, a recombinant baculovirus expressing glycoprotein gB of pseudorabies virus induces antibodies against gB protein in mice, suggesting that this recombinant baculovirus could be a vaccine candidate for pseudorabies (Aoki, 1999). More recently, Facciabene et al. demonstrated that the intramuscular injection of a baculovirus expressing CEA induces measurable CEA-specific CD4+ T-cell response (Facciabene, 2004). The immunogenic property of baculovirus is not restricted to CEA because the intramuscular injection of another baculovirus (Bac-E2) expressing E2 glycoprotein of HCV induces anti-E2 CD8+ T-cell response (Facciabene, 2004). Interestingly, when Bac-E2 is pseudotyped to display VSVG on the envelope, the minimal dose required to elicit a measurable T-cell response is 10-fold less, indicating that the VSVG-pseudotyped Bac-E2 appears to be a more potent vaccine vehicle than the unmodified virus.

It is also likely that baculovirus elicits an immune response against an antigen when it is displayed on the viral surface, because the immunodominant antigenic site of foot-and-mouth disease virus (FMDV) displayed on the baculovirus envelope preserves the antigenic epitopes on the virus envelope (Tami, 2000) and binds a panel of FMDV-specific MAb. A more recent study further shows that immunization with adjuvant-free baculovirus displaying rodent malaria Plasmodium berghei circumsporozoite protein (PbCSP) on the envelope induces high levels of antibodies and IFN-γ-secreting cells against PbCSP, and protects 60% of mice against sporozoite challenge (Yoshida, 2003).

Conventional influenza vaccines are based on inactivated viruses which are produced in embryonated eggs and inactivated by chemical means. The basis of protective humoral immunity is the development of neutralizing antibodies against the two major surface proteins, HA and NA. Antibody against HA generally neutralizes viral infectivity, by interfering either with viral attachment to sialic-acid receptors on the host cell surface or with fusion between viral and endosomal membrane (Brown, 1992). By contrast, anti-NA antibody does not prevent infection, but can reduce viral replication below a pathogenic threshold so that infection can occur without frank disease, by inhibiting the release of progeny virus from infected cells or by aggregating viral particles (Johansson, 1998). However, the virulent nature of the virus requires that the viruses be handled under biosafety level (BSL)-3 conditions, the production of vaccine candidates using conventional techniques would therefore require significant changes in current manufacturing procedures that might delay vaccine production in case of pandemic situation (Treanor, 2001). Besides, the 1997 Hong Kong virus could not be grown in embryonated eggs because it is too pathogenic and kills the embryos before high levels of virus are produced (Takada, 1999). Another disadvantage of the inactivated virus vaccine is that it also induces immune responses to the group-specific influenza A nucleoprotein (NP) antigen, which interferes with surveillance by prohibiting direct serological distinction of vaccinated from field exposed birds (Beard, 1991; Lee, 2004).

Several recombinant fowlpox viruses expressing the H5 antigens have been developed (Beard, 1991; Swayne, 2000a; Swayne, 2000b; Swayne, 1997), and one has been licensed and is currently being used in Mexico (Swayne, 1997). Experimental data have also been obtained for recombinant fowlpox virus expressing the H7 antigen (Boyle, 2000). Other vectors (e.g. infectious laryngotracheitis virus [Luschow, 2001]) have been used to successfully deliver the H5 or H7 antigens. The only field experience with the recombinant virus vector to control AI is obtained in Mexico, where fowlpox virus is used in the vaccination campaign against H5N2 virus. However, due to pre-existing immunity the vaccine does not induce protective immunity in birds that have had field exposure to the virus (Swayne, 2000a). Since serological positivity to fowlpox virus is widespread (due to field exposure and vaccination) in the poultry population, and can be in some instances unpredictable, the use of fowlpox virus vaccine is limited to a population that is sero-negative to the vector virus. No such vaccine has been licensed in the EU to date.

An alternative vaccine is a subunit vaccine that avoids all of the above pitfalls. Since the protection provided by influenza vaccines is based on the induction of virus neutralizing antibodies, mainly against HA, HA protein is an ideal target due to its strong immogenicity (Treanor, 2001; Crawford, 1999). However, HA is a membrane protein that is highly hydrophobic, the low solubility increases the difficulty in handling and reduces its effectiveness as a vaccine (Treanor, 2001). Using eukaryotic expression systems for HA production, the yield is low because of strong association of the protein with membranes (Hu, 2006). Moreover, the purification of recombinant HA is difficult owing to the low solubility.

A recent development in the production of H5N1 vaccine is the use of reverse genetics. Vaccine viruses obtained by reverse genetics contain the HA and NA genes of naturally circulating isolates in a genetic background of strains with high egg-growth capacity, and thus allow a well-matched nonpathogenic H5N1 vaccine to be produced rapidly (Webby, 2004). However, the possibility of re-assortment among field and vaccine virus RNA segments exists (Desselberger, 1978).

### SUMMARY OF THE INVENTION

The current invention relates to vaccines that use baculovirus vectors to expose a host organism to an immunogen, thereby eliciting an immune response. Baculovirus vectors can be induced to incorporate exogenous proteins into the viral envelope when specific signal peptides and protein domains are expressed with the exogenous protein in the form of a fusion protein. When the appropriate fusion protein is expressed, the protein is translocated to the plasma membrane where it is incorporated into the viral envelope upon virus budding.

The object of the current invention is to display HA on the baculoviral envelope by expressing it as a fusion protein with heterologous or homologous cytoplasmic domains. One embodiment of the current invention comprises a baculovirus vector which contains recombinant DNA encoding a signal peptide, the hemagglutinin immunogen, and the C-terminal domain of the gp64 protein, a native baculovirus envelope protein. The signaling peptide has the ability to direct the fusion protein to the cell membrane, while the C-terminal domain of the gp64 protein has the ability to direct the incorporation of the gp64 protein into the budding virion resulting in display of the fusion protein on the viral envelope.

### BRIEF DESCRIPTION OF DRAWINGS

FIG. 1 shows a schematic illustration of two-stage PCR;

FIG. 2 Shows a schematic illustration of pBac-HA and pBac-HA64 plasmids;

FIG. 3 Shows a confirmation of the expression of His₆-tagged rHA and rHA64 in Sf-9 cells;

FIG. 4 Shows a visualization of protein anchoring on the surface of plasma membrane;

FIG. 5 Shows an immunogold electron microscopic examination of Bac-CE, Bac-HA64 and Bac-HA;

FIG. 6 Shows the incorporation of recombinant HA into baculoviral envelope;

FIG. 7 Shows the transduction of mammalian cells with pseudotyped or non-pseudotyped baculoviruses;

FIG. 8 Shows the stability of pseudotyped and non-pseudotyped baculoviruses; and

FIG. 9 Shows hemagglutination inhibition (HI) titers in BALB/c mice immunized with various immunogens.

### DETAILED DESCRIPTION OF THE PREFERRED EMBODIMENT

As mentioned above, highly pathogenic AI virus of the H5 subtype has imposed a tremendous threat to the global public health. For effective control of global AI pandemic, the best way to keep AI virus out of humans is to keep it out of birds (Normile, 2005). Given the fact that baculovirus efficiently transduces mammalian cells and pseudotyped baculovirus is a promising vaccine vehicle, the primary objective of this invention is to develop pseudotyped baculovirus as a novel vaccine vehicle against AI virus infection. Because HA is the primary immunogen eliciting immune responses, this application aims at constructing pseudotyped baculovirus with HA (derived from H5 subtype) displayed on the envelope, in a hope that the native conformation of HA is retained for elicitation of neutralizing antibodies. To ensure that HA is efficiently incorporated, two recombinant baculoviruses were constructed, Bac-HA expressing HA fused with the cytoplasmic domain (CTD) derived from HA, and Bac-HA64 expressing HA fused with the CTD derived from gp64 (gp64 is the natural baculovirus envelope protein and its CTD may allow for more efficient incorporation of HA into baculovirus).

### Construction of Recombinant Baculoviruses Bac-HA and Bac-HA64

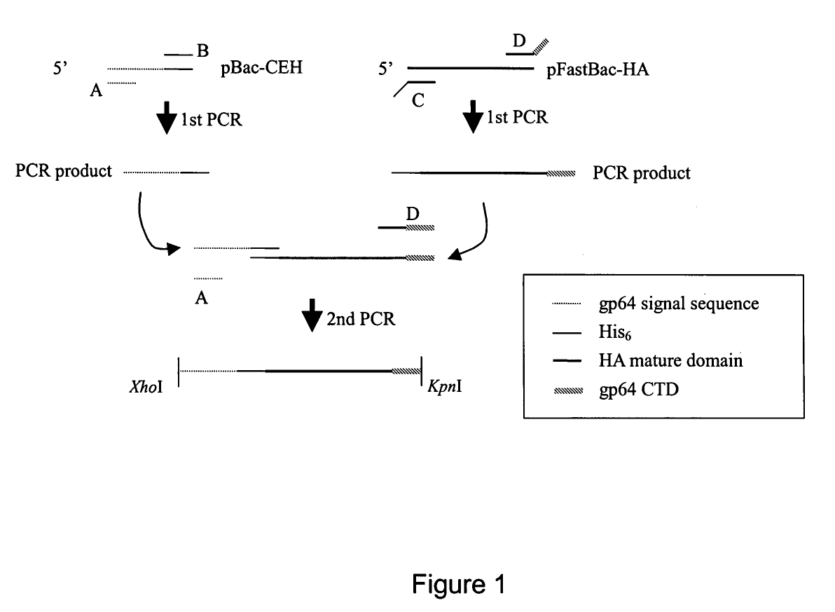

The full-length gene fragment encoding HA of AI virus (A/chicken/Taiwan/1209/03, H5N2) was cloned by polymerase chain reaction (PCR) into pFastBac-HA plasmid to express the recombinant HA protein in insect cells (Hu, 2006). In this study, the aim was to effectively display HA on the baculovirus envelope, therefore, an additional signal sequence derived from baculovirus envelope protein gp64 was genetically fused to the N-terminus of HA gene via a two-stage PCR approach (Hu, 2003a). Specifically, the wild-type gp64 gene was previously amplified by PCR from AcMNPV DNA and subcloned to pBac-CEH plasmid, thereby a stretch of 6 histidine residues (His₆ tag) was inserted between the signal sequence and the mature domain (Hu, 2003a). Five primers (A, B, C, D and E as shown in Table 1) were designed to construct the fusion genes using pBac-CEH and pFastBac-HA plasmids as the templates. Primers A (SEQ ID NO:1) and B (SEQ ID NO:2) were used to amplify the gp64 signal sequence (SS) while adding a His₆ tag at the C-terminus. Primers C (SEQ ID NO:3) and D (SEQ ID NO:4) (or E [SEQ ID NO:5]) were used to amplify the mature domain (MD, including the ectodomain and transmembrane (TM) domain) of HA, while adding a His₆ tag at the N-terminus and a cytoplasmic domain (CTD) at the C-terminus. Owing to the significance of CTD on the recognition with nucleocapsid and subsequent protein incorporation, two versions of CTD, one derived from gp64 (using primer D) and the other derived from HA (using primer E, not shown in FIG. 1), were fused to C-terminus. The resultant PCR products were mixed and subject to the second PCR to amplify the whole fusion: SS-His₆-MD-CTD, using primers A and D (or E). The two-stage PCR procedure is illustrated in FIG. 1. FIG. 1 shows the generation of the fusion gene SS-His₆-MD-gp64 CTD as an example. The gene fragments encoding gp64 SS-Hi₆ (left) and His₆-MD-CTD (right) were amplified in the first PCR stage. The two fusion gene fragments were then fused in the second PCR stage. Primers A and D were deigned to add restriction enzyme sites ^{Xho}I and ^{Kpn}I.

TABLE 1
Primers used in the cloning experiments.

| Primer | Orientation | Primer sequence |
|---|---|---|
| A | Forward | 5′-ATATCTCGAGATGCTACTAGTAAATCAGTCACACCAAGGCTTC |
| B | Reverse | 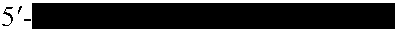 |
| C | Forward | 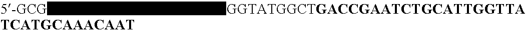 |
| D | Reverse | 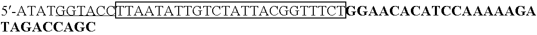 |
| E | Reverse | 5′-TTCAGTTAGCCTCCCCCATC |

Table 1: Primers used in cloning experiments. Primers A and B were used to amplify the gp64 signal sequence (SS) (sequence in bold) while adding a His₆ tag (shaded sequences). Primers C and D were used to amplify the mature domain (MD, consisting of the ectodomain and transmembrane domain) of HA, while adding a His₆ tag at the N-terminus and a cytoplasmic domain (CTD, boxed region) derived from gp64 at the C-terminus. Primers C and E were employed to amplify the MD and CTD of HA. The 5′ end of primers A and D (and E) were designed to harbor restriction enzyme sites Xho I and Kpn I, respectively, for the enzyme digestion after PCR amplification.

Percentage identity of two nucleic acid sequences is calculated by first aligning the first sequence with the second sequence, inserting gaps in the first sequence where insertions have occurred in the second sequence, and inserting gaps in the second sequence where deletions have occurred in the second sequence. The aligned nucleotides are then compared between the first sequence and the second sequence from the first position to the final position. Gaps, insertions, deletions or introns are not compared. The number of identical aligned nucleotides is then divided by the total number of aligned nucleotides to give the percent identity.

Percentage identity of two amino acid sequences is calculated by first aligning the first sequence with the second sequence, inserting gaps in the first sequence where insertions have occurred in the second sequence, and inserting gaps in the second sequence where deletions have occurred in the second sequence. The aligned amino acids are then compared between the first sequence and the second sequence from the first position to the final position. Gaps, insertions, deletions are not compared. The number of identical aligned amino acids is then divided by the total number of aligned amino acids to give the percent identity.

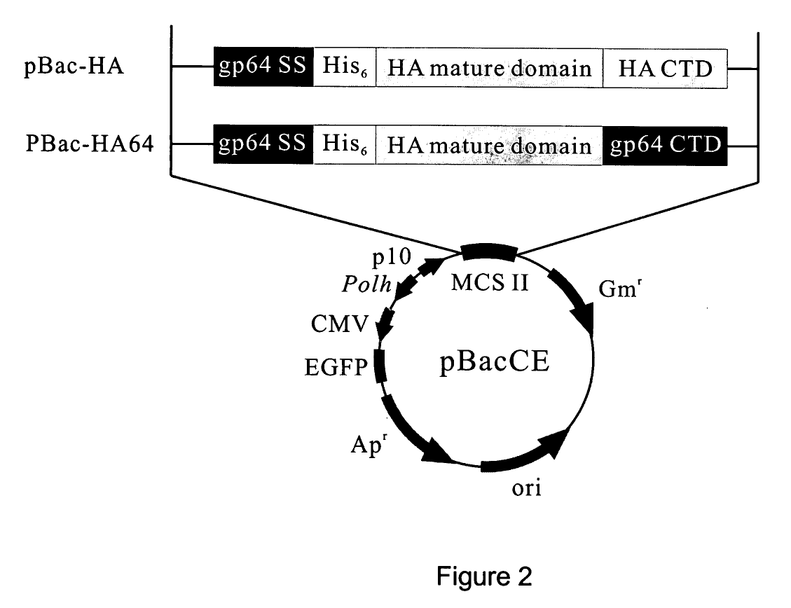

Additionally, primers A and D (or E) were designed to add restriction enzyme sites Kpn I and Xho I which allowed for insertion of the fusion gene into KpnI/XhoI site of MCS II of pBac-CE plasmid, under the transcriptional control of p10 promoter. pBac-CE was constructed in our lab previously using pFastBac™ DUAL (Invitrogen) as the backbone (Hu, 2003b) and harbored egfp (encoding enhanced green fluorescent protein, EGFP) gene under the transcriptional control of cytomegalovirus immediate early (CMV) promoter. The expression of EGFP served as a reporter for monitoring baculovirus transduction of mammalian cells. The resultant plasmids were designated pBac-HA and pBac-HA64 (FIG. 2). FIG. 2 shows a schematic illustration of pBac-HA and pBac-HA64 plasmids. The fusion gene fragments generated by two-stage PCR as shown in FIG. 1 were cloned into the ^{Kpn}I/^{Xho}I site of MCS II of pBac-CE plasmid, under the transcriptional control of p10 promoter. EGFP expression was controlled by CMV promoter as a reporter.

The recombinant baculoviruses were subsequently generated using Bac-to-Bac® system (Invitrogen) and designated Bac-HA and Bac-HA64, respectively. Bac-HA was designed to express His₆-tagged rHA (recombinant HA fused with gp64 SS at the N-terminus and HA CTD at the C-terminus). Bac-HA64 was designed to express rHA64 (recombinant HA fused with gp64 SS at the N-terminus and gp64 CTD at the C-terminus). Ideally, during virus replication the gp64 signal sequence directs the translocation of the chimeric protein to the insect cell plasma membrane and is cleaved after protein anchoring thus exposing His₆ tag to the outer surface. The transmembrane domain enables the protein to anchor on the plasma membrane, while the CTD mediates (1) the recognition of budding baculovirus nucleocapsid with the membrane-bound protein and (2) the incorporation of the anchored protein into the virus envelope.

The subsequent recombinant virus selection, plaque purification and amplification were performed using insect (Sf-9) cell as the host as described earlier (Hu, 2003a). The Sf-9 cells were maintained using TNM-FH medium supplemented with 10% fetal bovine serum. The titers of recombinant baculoviruses were determined by endpoint dilution method (Hu, 2003a) and are expressed as plaque forming units per milliliter (PFU/ml).

### Confirmation of Recombinant Protein (rHA and rHA64) Expression in Insect Cells

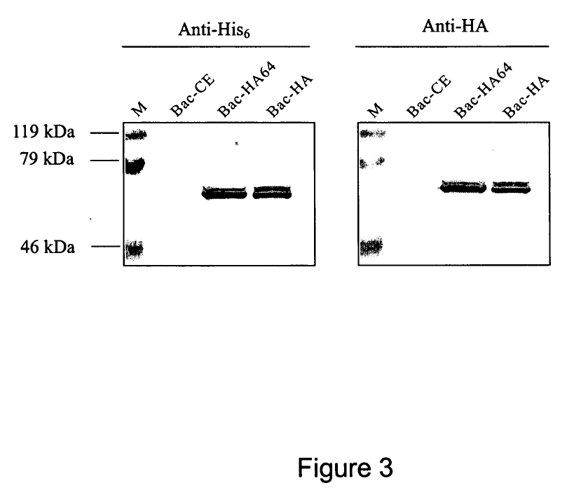

To confirm the expression of His₆-tagged rHA and rHA64 in insect (Sf-9) cells, the cells were respectively infected by Bac-CE (a control baculovirus expressing no HA [Hu, 2003b]), Bac-HA and Bac-HA64 at a multiplicity of infection (MOI) of 10. The cells were harvested at 3 days post-infection (dpi) and lysed in phosphate buffered-saline (PBS). The lysates were subject to SDS-PAGE, followed by Western blot analysis using anti-HA serum and anti-His₆ monoclonal antibody (MAb) as the primary antibodies. As shown in FIG. 3, the cells were infected by Bac-CE, Bac-HA and Bac-HA64 at MOI 10, harvested at 3 dpi and lysed in PBS. The lysates were subject to SDS-PAGE, followed by Western blot analysis. SDS-PAGE was performed by mixing cell lysate with sample buffer (0.5 M Tris-HCl, pH 6.8, 10% glycerol, 5% SDS, 5% β-mercaptoethanol, 0.25% bromophenol blue), boiling at 100° C. for 3 minutes, centrifuging for 1 minute, and loading onto a 10% slab gel. After electrophoresis, the proteins on the gel were transferred onto a nitrocellulose membrane following standard procedures (Bio-Rad Laboratories). Two primary antibodies were used for Western blot: chicken anti-HA polyclonal antibody and mouse anti-His₆ MAb (Invitrogen). The secondary antibodies were goat anti-checken and goat anti-mouse MAb conjugated with alkaline phosphatase (Kirkegaard and Perry Laboratories, KPL, Gaithersburg, Md.). The color development was performed by immersing the membrane in BCIP/NBT reagent (Sigma). As shown in FIG. 3, the cells infected by Bac-CE expressed no HA, whereas the cells infected by either Bac-HA or Bac-HA64 expressed a ≈69 kD protein that was detected by both anti-His₆ and anti-HA antibodies, thus confirming the expression of His₆-tagged rHA and rHA64.

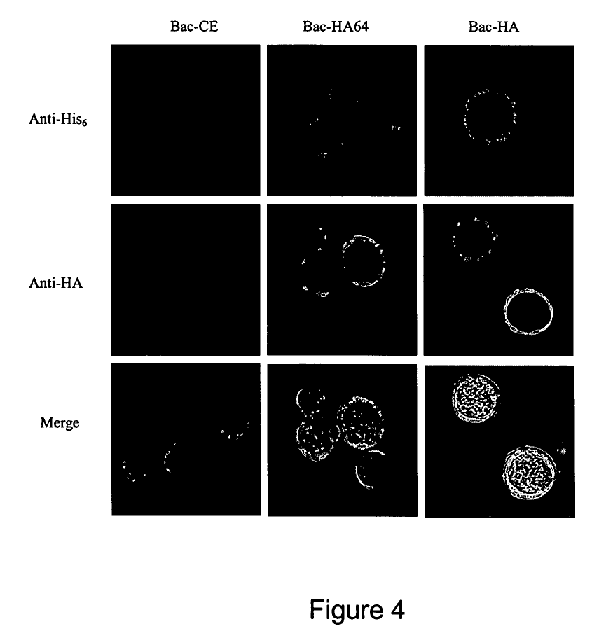

To confirm whether the His₆-tagged rHA and rHA64 were properly translocated to the surface of plasma membrane, the cells were cultured on sterile cover slips, infected by Bac-CE, Bac-HA64 or Bac-HA, and subject to immunofluorescence labeling/confocal microscopy visualization at 2 dpi (FIG. 4). As shown in FIG. 4, the Sf-9 cells were cultured on sterile cover slips (placed in 6-well plates) and infected by Bac-HA and Bac-HA64 at MOI 10. Two days later, the medium was removed, and the cells were fixed by methanol/acetone (1:1) for 5 min at −20° C., rinsed with 1 ml of PBS and then blocked with 2% bovine serum albumin in PBS (1 ml) for 30 min at 37° C. The cells were then incubated with the primary antibody (mouse anti-His₆ MAb, 1:300 dilution, Amersham Biosciences) for 1 h at 37° C., followed by PBS washes 3 times. The cells were then incubated with the secondary antibody (FITC-conjugated anti-mouse MAb, 1:50 dilution, KPL) for 1 h at 37° C., followed by 3 PBS washes. Protein localizations were visualized by a confocal microscope (TCS SP2, Leica, Germany). His₆-tagged proteins emitted green fluorescence under the microscope. The merged photographs illustrate the colocalization of rHA and rHA64 with plasma membrane.

Both Bac-HA and Bac-HA64 expressed proteins that were detected by anti-His₆ and anti-HA antibodies. These proteins co-localized with the plasma membrane, indicating that both rHA and rHA64 were translocated and anchored on the plasma membrane.

### Display of rHA and rHA64 on the Baculoviral Envelope

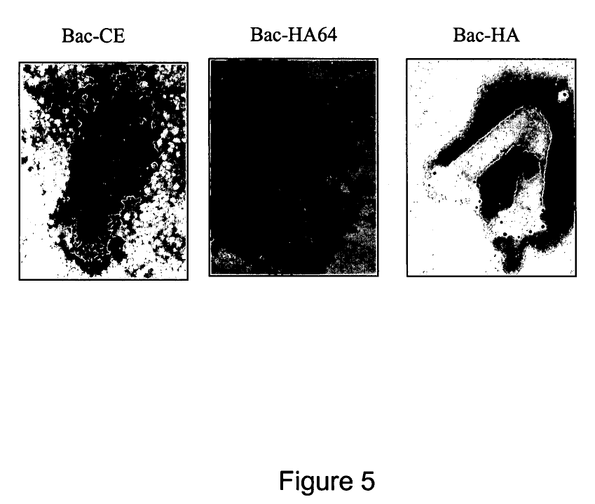

To examine whether the rHA (rHA64) anchored on the plasma membrane was successfully displayed on the baculoviral envelope, Bac-HA, Bac-HA64 and Bac-CE were purified by sucrose gradient ultracentrifugation and subject to immunogold electron microscopy using anti-His₆ antibody as the primary Ab. As shown in FIG. 5, the recombinant viruses were produced by infecting insect cells (Sf-9) at MOI 0.1 and harvested at 4 days post-infection. The virus supernatant was individually purified by sucrose gradient ultracentrifugation (O'Reilly, 1992). Briefly, 33 ml virus supernatant was loaded to each ultracentrifugation tube (Hitachi, 40PA) and then underlaid with 3 ml sucrose solution (25% (wt/wt), dissolved in phosphate buffered saline). The virus was pelleted by ultracentrifuation at 90,000×g (CP100MX, Hitachi) for 1.5 hr, and the pellets were pooled and resuspended with 5 ml PBS. For further virus purification, the concentrated viruses (0.5-1 ml) were loaded onto a sucrose gradient (10 ml) consisting of four layers of 35, 43, 55 and 60% (wt/wt) sucrose solution. After centrifugation at 90000×g for 3 h, baculovirus that sedimented near 43-47% sucrose was collected by syringe, diluted in PBS and ultracentrifuged (90,000×g for 90 min) again. The purified virus pellet was resuspended in PBS and then was subject to immunogold electron microscopy as follows. The carbon-coated copper grid (PELCO) was floated on top of 10 μl purified baculovirus solution for 30 min. The grid was then floated on top of a drop of blocking solution (PBS containing 1% BSA and 25 mM glycine pH 6.2) for 20 min and washed with PBS 3×5 min. Following the washing step, the grid was transferred onto a drop (15 μl each) of the anti-His₆ MAb solution (1:100 diluted) and incubated for 30 min at room temperature. After two PBS washes, the grid was then floated on the anti-mouse MAb conjugated with 5-nm gold particle (1:50-dilution, Sigma) for 30 min at room temperature. After washing with PBS, the grid was negatively stained with 2% phophotungstic acid (PTA, Sigma) for 2 min and air-dried at room temperature. The gris were examined under transmission electron microscope (Hitachi, H-7500).

FIG. 5 reveals no gold particles on the surface of Bac-CE, whereas gold particles were evidently observed on the surface of both Bac-HA64 and Bac-HA, indicating the incorporation and display of rHA64 and rHA on the viral envelope.

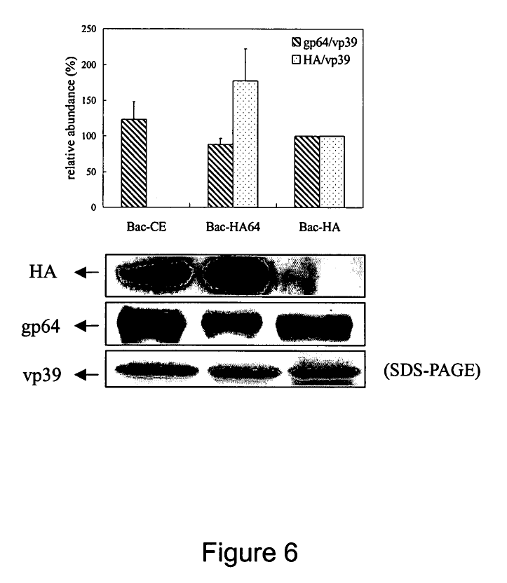

The differential incorporation of rHA64 and rHA into the purified baculoviruses (Bac-HA64 and Bac-HA) was visualized by SDS-PAGE/Western blot and quantified by scanning densitometry (FIG. 6) by which the amounts of His₆-tagged HA and gp64 incorporated into the viruses were normalized based on equal amounts of vp39 (the major capsid protein). As shown in FIG. 6, the purified viruses (Bac-CE, Bac-HA64 and Bac-HA) were subject to SDS-PAGE and Western blot using anti-HA or anti-gp64 Ab as the primary antibodies. The band intensities for HA and gp64 on the Western blot, as well as the band intensities for vp39 on the SDS-PAGE, were scanned and analyzed by Scion Image Shareware. Compared to Bac-HA, Bac-HA64 incorporated ≈77% more His₆-tagged HA into the virus, but contained virtually equal amounts of gp64. Conversely, Bac-CE contained no HA, but incorporated ≈24% more gp64 than Bac-HA.

### The Choice of CTD Influenced the Baculovirus-Mediated Expression

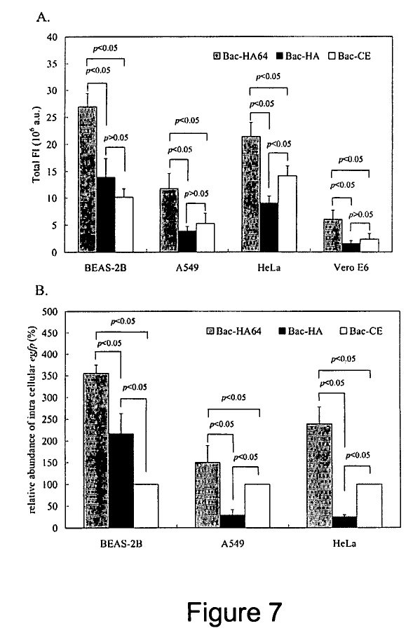

Whether the choice of CTD influenced the ability of baculovirus to transduce mammalian cells was further examined. Toward this end, four different mammalian cell lines (BEAS-2B, A549, HeLa and Vero E6) were transduced by Bac-HA, Bac-HA64 or the control virus Bac-CE using the same virus dosage (MOI 35). The cells were harvested at 1 day post-transduction and analyzed for total FI by flow cytometry (FIG. 7A). Among these 3 viruses, Bac-HA64 resulted in significantly higher EGFP expression levels in all 4 cell lines. Compared with Bac-CE, however, Bac-HA led to similar EGFP expression levels (p>0.05) in BEAS-2B, A549 and Vero E6 cells, and inferior EGFP expression (p<0.05) in HeLa cells.

The amounts of intracellular egfp genes within BEAS-2B, A549 and HeLa cells were also quantified at 1 day post-transduction by Q-PCR. For each cell line, the quantities of egfp genes carried by Bac-HA and Bac-HA64 were normalized against that carried by Bac-CE (FIG. 7B). In comparison with Bac-CE, the amounts of intracellular egfp delivered by Bac-HA64 significantly excelled in all 3 cell lines (p<0.05). Conversely, the amounts of intracellular egfp delivered by Bac-HA were only higher in BEAS-2B (p<0.05), but were lower in A549 and HeLa cells (p<0.05). These data indicate that Bac-HA64 yielded improved transduction whereas. Bac-HA did not.

### The Amounts of Ha Incorporated Influenced the Baculovirus Stability

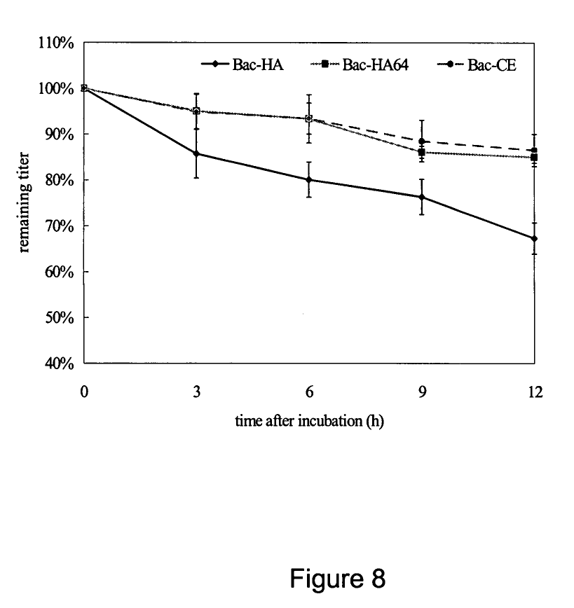

To examine whether the incorporated HA via different CTD affected the virus stability, viruses (Bac-CE, Bac-HA64 and Bac-HA) of identical concentrations (5×10⁷ pfu/ml) were incubated at 37° C. and the remaining titers were monitored. As shown in FIG. 8, Baculovirus supernatants (Bac-CE, Bac-HA64 and Bac-HA) of identical concentrations (5×10⁷ pfu/ml) were incubated at 37° C. and the decay of virus transducing titer was monitored. Briefly, the virus supernatants sampled at various times were diluted 2-fold serially in TNM-FH medium in microfuge tubes. One hunderd microliter diluted virus solutions were directly used to transduce HeLa cells as described (Chan, 2006). At 24 hr post-transduction, the cells were trypsinized and analyzed for the transduction efficiencies and mean fluorescence intensity by flow cytometry. The percentage of GFP-emitting cells was determined for each viral dilution. The transducing titers were calculated according to the following equation (Transfiguracion, 2003; Logan, 2004) and are expressed as transducing units per milliliter (TU/ml):

Transducing titer (TU/ml)=[(% GFP+cells)/100]×dilution factor×cell number×reciprocal of volume(ml)

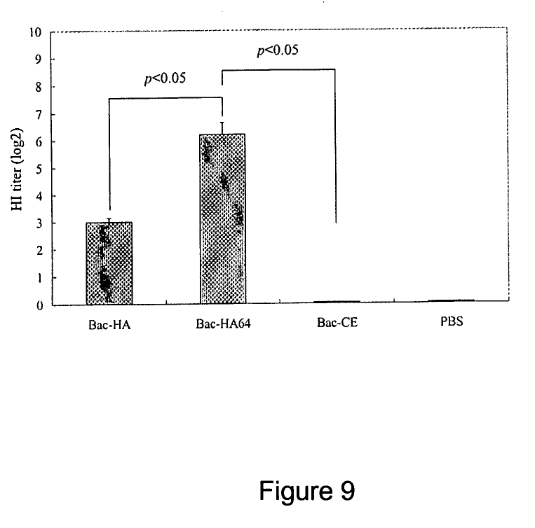

FIG. 9 depicts that both Bac-HA64 and Bac-CE exhibited comparably higher tolerance to the incubation at 37° C., as ≈88% of transducing titer was retained after 12 h. However, the titer of Bac-HA dropped to ≈69% of the original after 12 h incubation, indicating the inferior stability of Bac-HA.

### Immunogen Eliciting Properties of HA-Pseudotyped Baculoviruses

To examine whether the HA displayed on baculoviral envelope could serve as the immunogen eliciting anti-HA antibodies in vivo, 2 groups of mice, each comprising 8 female BALB/c mice (6 weeks old, purchased from National Experimental Animal Center), were immunized intraperitoneally (Facciabene, 2004) with 1.5×10⁸ PFU purified Bac-HA and Bac-HA64, respectively. As negative controls, five mice were injected with purified Bac-CE or PBS. For each mouse, the purified virus (250 μl) was mixed with 250 μl complete Freund' adjuvant for injection. Two weeks after the primary injection, the mice received one booster shot using equal amount of the purified virus (1.5×10⁸ PFU/mice) with the incomplete Freund' adjuvant. Blood samples were taken from the tail vein on week 6 and heat inactivated.

Whether the serum antibodies were capable of inhibiting hemagglutination were determined by hemagglutination inhibition (HI) assay (Coligan, 2005). As shown in FIG. 8, Bac-CE, Bac-HA and Bac-HA64 were purified by ultracentrifugation as described above. Two groups of mice, each comprising 8 female BALB/c mice (6 weeks old, purchased from National Experimental Animal Center), were immunized intraperitoneally (Facciabene, 2004) with 1.5×10⁸ PFU of purified Bac-HA and Bac-HA64, respectively. As negative conrols, 5 mice were injected with purified Bac-CE or PBS. One of skill in the art would recognize that a variety of means for introducing the vaccine into the host exist, including subcutaneous injection, intravenous injection, intra-arterial injection, electroporation, and transdermal administration, among others. For each mouse, the purified virus (250 μl) was mixed with 250 μl complete Freund's adjuvant for injection. Two weeks after the primary injection, the mice received one booster shot using equal amount of the purified virus (1.5×10⁸ PFU/mice) with the incomplete Freund' adjuvant. Blood was taken from the tail vein on week 6. The collected blood samples were left at room temperature for 1 h, centrifuged at 1000×g for 5 min to remove cells, and incubated at 56° C. for 30 min to inactivate complement proteins. The sera were stored at −80° C. until analysis. Whether the serum antibodies were capable of inhibiting hemagglutinin were titered by hemagglutination inhibition (HI) assay (Coligan, 2005). The hemagglutination unit of the inactivated AIV were first measured according to the standard protocols (Coligan, 2005).

As shown in FIG. 9, the negative control Bac-CE and PBS induced no HI titers while both Bac-HA and Bac-HA64 induced HI titers. Intriguingly, immunization with Bac-HA64 elicited significantly higher HI titers compared to Bac-HA. These data indicated that the incorporated HA successfully induced functional antibodies, and the amount of antibody was correlated with the amount of HA incorporated.

Although baculovirus displaying immunogen as a vaccine candidate has been proposed, this is the first report demonstrating that HA displaying baculovirus can be used as a vaccine to induce antibody responses to AI virus.

Normally other researchers pseudotype the baculovirus simply using the envelope protein (e.g. VSV-G) and the homologous CTD (e.g. CTD derived from VSV-G). However, here we demonstrate that, the way HA is displayed on the baculoviral envelope has a tremendous impact on the virus property and vaccine efficacy. The CTD derived form the baculovirus endogenous gp64 endows more efficient incorporation of HA protein (compared to the HA CTD) into the baculovirus, which in turn, results in the display of more HA on baculoviral envelope and confers higher immunogenicity.

As a vaccine vehicle, baculovirus possesses the following advantages over other vaccine types: (1) Baculovirus is non-pathogenic to humans, thus obviating BSL-3 facilities for vaccine production. (2) Baculovirus can be easily produced to high titers simply by infecting natural host insect cells, thus facilitating large-scale production. (3) Recombinant baculovirus vaccine enables the differentiation between infected and vaccinated animals, because it does not induce the production of antibodies against the nucleoprotein antigen, which is common to all AI viruses. (4) Baculovirus vaccine should avoid pre-existing immunity problem (i.e. humans do not have anti-baculovirus antibodies that can immediately inactivate baculovirus.

This platform can be extended to construct other pseudotyped baculoviruses. For example, we may replace EGFP with neuraminidase (NA) to construct a virus displaying HA and capable of expressing NA in the animals. The HA displayed on baculovirus envelope supposedly retains the native conformation for eliciting the humoral immunity, and the NA expressed in animals supposedly undergoes accurate post-translational modifications. Since AI virus infection poses a tremendous threat to poultry industry and global public health, the application of recombinant baculovirus-based vaccine provides an alternative promising strategy to prevent avian flu epidemic and will contribute greatly to the development of anti-AI vaccine.

As AI infection has turned into a tremendous threat to humans around the globe, the development of AI vaccine has become imminent. The approval of this novel vaccine platform can open a new avenue to vaccine development and provide an alternative option to existing vaccine technology. It is expected that this invention can result in a rapid technology transfer.

The term “conservatively modified variations” of a particular nucleic acid sequence refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given polypeptide. For instance, the codons CGU, CGC, CGA, CGG, AGA, and AGG all encode the amino acid arginine. Thus, at every position where an arginine is specified by a codon, the codon can be altered to any of the corresponding codons described without altering the encoded polypeptide. Such nucleic acid variations are “silent variations,” which are one species of “conservatively modified variations.” Every nucleic acid sequence herein which encodes a polypeptide also describes every possible silent variation. One of skill will recognize that each codon in a nucleic acid (except AUG, which is ordinarily the only codon for methionine) can be modified to yield a functionally identical molecule by standard techniques. Accordingly, each “silent variation” of a nucleic acid which encodes a polypeptide is implicit in each described sequence. Furthermore, one of skill will recognize that individual substitutions, deletions or additions which alter, add or delete a single amino acid or a small percentage of amino acids (typically less than 15%, more typically less than 5%, and even more typically less than 1%) in an encoded sequence are “conservatively modified variations” where the alterations result in the substitution of an amino acid with a chemically similar amino acid. Conservative amino acid substitutions providing functionally similar amino acids are well known in the art. Every amino acid sequence herein also describes every possible variation containing one or more conservative amino acid substitution. The following six groups each contain amino acids that are generally considered conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

### REFERENCES CITED

The following U.S. Patent documents and publications are hereby incorporated by reference.

### PUBLICATIONS

Abe, T, Takahashi, H, Hamazaki, H, Miyano-Kurosaki, N, Matsuura, Y, Takaku, H. Baculovirus induces an innate immune response and confers protection from lethal influenza virus infection in mice. J Immunol 2003; 171: 1133-1139.
Alexander, D J. A review of avian influenza in different bird species. Vet Microbiol 2000; 74: 3-13.
Aoki, H, Sakoda, Y, Jukuroki, K, Takada, A, Kida, H, Fukusho, A. Induction of antibodies in mice by a recombinant baculovirus expressing pseudorabies virus glycoprotein B in mammalian cells. Vet Microbiol 1999; 68: 197-207.
Barsoum, J, Brown, R, McKee, M, Boyce, F M. Efficient transduction of mammalian cells by a recombinant baculovirus having the vesicular stomatitis virus G glycoprotein. Hum Gene Ther 1997; 8: 2011-2018.
Beard, C W, Schnitzlein, W M, Tripathy, D N. Protection of chickens against highly pathogenic avian influenza virus (H5N2) by recombinant fowlpox viruses. Avian Dis 1991; 35: 356-359.
Boublik, Y, Di Bonito, P, Jones, I M. Eukaryotic virus display: engineering the major surface glycoprotein of the Autographa californica nuclear polyhedrosis virus (AcNPV) for the presentation of foreign proteins on the virus surface. Biotechnology 1995; 13: 1079-1084.
Boyle, D B, Selleck, P, Heine, H G Vaccinating chickens against avian influenza with fowlpox recombinants expressing the H7 haemagglutinin. Austral Vet J 2000; 78: 44-48.
Brown, D W, Kawaoka, Y, Webster, R G, Robinson, H L. Assessment of retrovirus-expressed nucleoprotein as a vaccine against lethal influenza virus infections of chickens. Avian Dis 1992; 36: 515-520.
Chan, Z-R, Lai, C-W, Lee, H-P, Chen, H-C, Hu, Y-C. Determination of the baculovirus transducing titer in mammalian cells. Biotechnol Bioeng 2006; 93: 564-571.
Coligan, J E, Bierer, B E, Margulies, D H, Shevach, E M, Strober, W. In Short protocols in immunology; John Wiley & Sons, 2005; 11-42-11-43.
Crawford, J, Wilkinson, B, Vosnesensky, A, Smith, G, Garcia, M, Stone, H, et al. Baculovirus-derived hemagglutinin vaccines protect against lethal influenza infections by avian H5 and H7 subtypes. Vaccine 1999; 17: 2265-2274.
Desselberger, U, Nakajima, K, Alfino, P, Pedersen, F S, Haseltine, W, Hannoun, C, et al. Biochemical evidence that ‘new’ influenza virus strains in nature may arise by recombination (reassortment). Proc Natl Acad Sci 1978; 75: 3341-3345.
Facciabene, A, Aurisicchio, L, La Monica, N. Baculovirus vectors elicit antigen-specific immune responses in mice. J Virol 2004; 78: 8663-8672.
Gronowski, A M, Hilbert, D M, Sheehan, K C F, Garotta, G, Schreiber, R D. Baculovirus stimulates antiviral effects in mammalian cells. J Virol 1999; 73: 9944-9951.
Ho, Y-C, Chen, H-C, Wang, K-C, Hu, Y-C. Highly efficient baculovirus-mediated gene transfer into rat chondrocytes. Biotechnol Bioeng 2004; 88: 643-651.
Hofmann, C, Sandig, V, Jennings, G, Rudolph, M, Schlag, P, Strauss, M. Efficient gene-transfer into human hepatocytes by baculovirus vectors. Proc Natl Acad Sci USA 1995; 92: 10099-10103.
Hu, Y-C, Luoa, Y-L, Jia, W-T, Chulua, J L C, Chang, P-C, Wang, C-Y, et al. Dual expression and analysis of the HA protein of H5N2 avian influenza virus. J Virol Methods 2006; Submitted:.
(Hu, 2003b) Hu, Y-C, Tsai, C-T, Chang, Y-J, Huang, J-H. Enhancement and prolongation of baculovirus-mediated expression in mammalian cells: focuses on strategic infection and feeding. Biotechnol Prog 2003; 19: 373-379.
(Hu, 2003a) Hu, Y-C, Tsai, C-T, Chung, Y-C, Lu, J-T, Hsu, JT-A. Generation of chimeric baculovirus with histidine-tags displayed on the envelope and its purification using immobilized metal affinity chromatography. Enzyme Microb Technol 2003; 33: 445-452.
Hu, Y-C. Baculovirus as a highly efficient expression vector in insect and mammalian cells. Acta Pharmacol Sin 2005; 26: 405-416.
Iwasaki, N, Yamane, S T, Majima, T, Kasahara, Y, Minami, A, Harada, K, et al. Feasibility of polysaccharide hybrid materials for scaffolds in cartilage tissue engineering: Evaluation of chondrocyte adhesion to polyion complex fibers prepared from alginate and chitosan. Biomacromolecules 2004; 5: 828-833.
Jarvis, D L, Garcia, A, Jr. Long-term stability of baculoviruses stored under various conditions. Biotechniques 1994; 16: 508-513.
Johansson, B, Grajower, B, Kilbourne, E. Infection-permissive immunization with influenza virus neuraminidase prevents weight loss in infected mice. Vaccine 1993; 11: 1037-1039.
Johansson, B E, Matthews, J T, Kilbourne, E D. Supplementation of conventional influenza A vaccine with purified viral neuraminidase results in a balanced and broadened immune response. Vaccine 1998; 16: 1009-1015.
Kost, T A, Condreay, J P. Recombinant baculoviruses as mammalian cell gene delivery vectors. Trends Biotechnol 2002; 20: 173-180.
Lee, C W, Senne, D A, Suarez, D L. Effect of vaccine use in the evolution of Mexican lineage H5N2 avian influenza virus. J Virol 2004; 78: 8372-8381.
Lee, C W, Senne, D A, Suarez, D L. Effect of vaccine use in the evolution of Mexican lineage H5N2 avian influenza virus. Journal of Virology 2004; 78: 8372-8381.
Lee, C W, Senne, D A, Suarez, D L. Generation of reassortant influenza vaccines by reverse genetics that allows utilization of a DIVA (Differentiating Infected from Vaccinated Animals) strategy for the control of avian influenza. Vaccine 2004; 22: 3175-3181.
Liu, J, Xiao, H, Lei, F, Zhu, Q, Qin, K, Zhang, X, et al. Highly pathogenic H5N1 influenza virus infection in migratory birds. Science 2005; 309: 1206.
Logan, A C, Nightingale, S J, Haas, D L, Cho, G J, Pepper, K A, Kohn, D B. Factors influencing the titer and infectivity of lentiviral vectors. Human Gene Ther 2004; 15: 976-988.
Luschow, D, Werner, O, Mettenleiter, T C, Fuchs, W. Protection of chickens from lethal avian influenza A virus infection by live-virus vaccination with infectious laryngotracheitis virus recombinants expressing the hemagglutinin (H5) gene. Vaccine 2001; 19: 4249-4259.
Monsma, S A, Oomens, A G P, Blissard, G W. The gp64 envelope fusion protein is an essential baculovirus protein required for cell-to-cell transmission of infection. J Virol 1996; 70: 4607-4616.
Mottershead, D G, Alfthan, K, Ojala, K, Takkinen, K, Oker-Blom, C. Baculoviral display of functional scFv and synthetic IgG-binding domains. Biochem Biophys Res Commun 2000; 275: 84-90.
Mounts, A W, Kwong, H, Izurieta, H S, Ho, Y, Au, T, Lee, M, et al. Case-control study of risk factors for avian influenza A (H5N1) disease, Hong Kong, 1997. J Infect Dis 1999; 180: 505-508.
Normile, D. Avian influenza: China will attempt largest-ever animal vaccination campaign. Science 2005; 310: 1256-1257.
Oomens, A G P, Monsma, S A, Blissard, G W. The baculovirus gp64 envelope fusion protein-synthesis, oligomerization, and processing. Virology 1995; 209: 592-603.
O'Reilly, D, Miller, L, Luckow, V In Baculovirus expression vectors: a laboratory manual; W.H. Freeman and Co, 1992; 109-215.
Raty, J K, Airenne, K J, Marttila, A T, Marjomaki, V, Hytonen, V P, Lehtolainen, P. et al. Enhanced gene delivery by avidin-displaying baculovirus. Molecular Therapy 2004; 9: 282-291.
(Swayne 2000a) Swayne, D E, Beck, J R, Kinney, N. Failure of a recombinant fowl poxvirus vaccine containing an avian influenza hemagglutinin gene to provide consistent protection against influenza in chickens preimmunized with a fowl pox vaccine. Avian Diseases 2000; 44: 132-137.
Swayne, D E, Beck, J R, Mickle, T R. Efficacy of recombinant fowl poxvirus vaccine in protecting chickens against a highly pathogenic Mexican-origin H5N2 avian influenza virus. Avian Diseases 1997; 41: 910-922.
(Swayne, 2000b) Swayne, D E, Garcia, M, Beck, J R, Kinney, N, Suarez, D L. Protection against diverse highly pathogenic H5 avian influenza viruses in chickens immunized with a recombinant fowlpox vaccine containing an H5 avian influenza hemagglutinin gene insert. Vaccine 2000; 18: 1088-1095.
Takada, A, Kuboki, N, Okazaki, K, Ninomiya, A, Tanaka, H, Ozaki, H, et al. Avirulent avian influenza virus as a vaccine strain against a potential human pandemic. J Virol 1999; 73: 8303-8307.
Tami, C, Farber, M, Palma, E L, Taboga, O. Presentation of antigenic sites from foot-and-mouth disease virus on the surface of baculovirus and in the membrane of infected cells. Arch Virol 2000; 145: 1815-1828.
Transfiguracion, J, Jaalouk, D E, Ghani, K, Galipeau, J, Kamen, A. Size-exclusion chromatography purification of high-titer vesicular stomatitis virus G glycoprotein-pseudotyped retrovectors for cell and gene therapy applications. Human Gene Therapy 2003; 14: 1139-1153.
Treanor, J J, Wilkinson, B E, Masseoud, F, Hu-Primmer, J, Battaglia, R, O'Brien, D, et al. Safety and immunogenicity of a recombinant hemagglutinin vaccine for H5 influenza in humans. Vaccine 2001; 19: 1732-1737.
van Loo, N D, Fortunati, E, Ehlert, E, Rabelink, M, Grosveld, F, Scholte, BJ. Baculovirus infection of nondividing mammalian cells: mechanisms of entry and nuclear transport of capsids. J Virol 2001; 75: 961-970.
Webby, R J, Perez, D R, Coleman, J S, Guan, Y, Knight, J H, Govorkova, E A, et al. Responsiveness to a pandemic alert: use of reverse genetics for rapid development of influenza vaccines. Lancet 2004; 363: 1099-1103.
WHO. Avian Influenza A (H5N1) Infection in Humans. N Engl J Med 2005; 353: 1374-1385.
Yoshida, S, Kondoh, D, Arai, E, Matsuoka, H, Seki, C, Tanaka, T, et al. Baculovirus virions displaying Plasmodium berghei circumsporozoite protein protect mice against malaria sporozoite infection. Virology 2003; 316: 161-170.

### WEBSITES

http://www.who.int/csr/disease/avian_influenza/country/cases_table_{—}2006_{—}01_{—}05/en/index.html
http://www.who.int/csr/disease/avian_influenza/country/cases_table_{—}2006_{—}01_{—}19/en/index.html

What is claimed is:

1. A recombinant DNA vector comprising:
a hemagglutinin sequence, being a first nucleotide sequence encoding a first polypeptide, the first polypeptide comprising an amino acid sequence being at least 80% identical to SEQ ID NO:1;
a signal peptide sequence, being a second nucleotide sequence encoding a second polypeptide, the second polypeptide comprising a membrane localization sequence; and
an incorporation sequence, being a third nucleotide sequence encoding a third polypeptide, the third polypeptide comprising a domain of a viral envelope glycoprotein capable of interacting with a viral nucleocapsid;
wherein the HA sequence, the signal peptide sequence, and the incorporation sequence are operably linked to an expression control sequence in an expression vector.

2. The recombinant DNA vector of claim 1, wherein the first polypeptide is at least 95% identical to SEQ ID NO:1.

3. The recombinant DNA vector of claim 1, wherein the first polypeptide is identical to SEQ ID NO:1.

4. The recombinant DNA vector of claim 1, wherein the first nucleic acid sequence is 95% identical to SEQ ID NO:2.

5. The recombinant DNA vector of claim 1, wherein the second nucleotide sequence is operably linked to the 5′-end of the hemagglutinin sequence in the same reading frame, and the second polypeptide comprises the N-terminal signal sequence of the gp64 protein.

6. The recombinant DNA vector of claim 1, wherein the third nucleotide sequence is operably connected to the 3′-end of the hemagglutinin sequence in the same reading frame, and the third polypeptide comprises the C-terminal domain of the gp64 protein.

7. The recombinant DNA vector of claim 1, wherein the third nucleotide sequence is operably connected to the 3′-end of the hemagglutinin sequence in the same reading frame, and the third polypeptide comprises the C-terminal domain of the hemagglutinin protein.

8. The recombinant DNA vector of claim 1, further comprising a fourth nucleotide sequence operably connected to the 5′-end of the hemagglutinin sequence in the same reading frame, the fourth nucleotide sequence encoding a fourth polypeptide, the fourth polypeptide comprising an epitope tag.

9. The recombinant DNA vector of claim 8, wherein the fourth polypeptide comprises 6 histidine residues.

10. The recombinant DNA vector of claim 1 wherein the expression vector is a baculovirus vector.

11. The recombinant DNA vector of claim 1, wherein the expression vector is a baculovirus vector derived from the pFastBac™ DUAL vector.

12. The recombinant DNA vector of claim 1, wherein the expression control sequence is a p10 promoter.

13. The recombinant DNA vector of claim 1, wherein the expression control sequence is a polyhedrin promoter.

14. A method for displaying an avian influenza antigen on a cell membrane of a host cell of a host or viral envelope of a virus, comprising:
introducing the recombinant DNA vector of claim 1 into the host.

15. The recombinant DNA vector of claim 14, wherein the avian influenza antigen is hemagglutinin.

16. The recombinant DNA vector of claim 14, wherein the virus is a baculovirus.

17. A method for inducing immunogenicity in a host comprising:
introducing the recombinant DNA vector of claim 1 into the host.

18. A method for increasing the titer of antibodies against an avian influenza antigen in a host, comprising:
introducing the recombinant DNA vector of claim 1 into the host.

19. The method of claim 18, wherein the avian influenza antigen comprises hemagglutinin.


SEQUENCE LISTING (7 sequences)

SEQ ID NO: 1 - protein, 564 aa, Artificial sequence
  Translation from mRNA of Influenza A virus H5N2
MERIVIAFAIISIVTGDRICIGYHANNSTKQVDTIMEKNVTVTHAQDILEKEHNGRLCSLKGVKPLILKDCSVAGWLLGNPMCDEFLNVPEWSYIVEKDKPANGLCYPGNFNYYEELKHLMSSTNHFEKIQIFPRSSWSNHDASSGVSSACPYNGRSSSFRNVVWLIKKNNVYRTITRTYHNTNVEDLLILWGIHHPNDAAEQIKLYQNPNTYVSVGTSTLNQRSIPEIATRPKVNGQSGRMEFFWTILRPNDSITFESTGNFIAPEYAYKIVKKGDSTIMKSELSYGNCDTKCQTPVGAINSSMPFHNVHPFTIGECPKYVKLKKLVLATGLRNVPQREKRGLFGAIAGFIEGGWQGMVDGWYGYHHSNEQGSGYAADKESTQKAVDGITNKVNSIIDKMNTQFEAVGKEFNNLERRIENLNKKMEDGFIDVWTYNAELLVLMENERTLDLHDSNVKNLYDKVRHQLRDNAKELGNGCFEFYHRCDDKCMESVRNGTYDYPQYSEESRLKREEIDGVKLESMGAYQILSIYSTVASSLALAIMVAGLSFWMCSNGSLQCRICI

SEQ ID NO: 2 - DNA, 1770 bp, Artificial sequence
  Origin of RNA from Influenza A virus H5N2
agcaaaagcaggggtataatctgatatcaaaatggaaagaatagtgattgcctttgcaataatcagcattgtcacaggtgaccgaatctgcattggttatcatgcaaacaattcaacaaaacaggttgacacaatcatggagaagaatgtgacggtcacacatgctcaggacatactggaaaaggaacacaatggaagactctgcagtcttaaaggagtgaagcccctcattctgaaggactgcagtgtagctggatggcttcttggaaatccaatgtgtgatgaattcctgaatgtaccggaatggtcatacattgtggaaaaagacaagccagccaatggcctgtgctatccgggaaacttcaactattatgaagaactgaagcatttaatgagcagcacaaaccattttgagaaaattcagatatttcctaggagctcttggtccaaccatgatgcctcatcaggagtgagctcagcatgcccatacaatggtaggtcttcctctttcaggaatgtagtgtggttgatcaagaagaacaatgtgtaccgaacaataacaaggacctaccacaacaccaatgtagaagaccttttaatattatggggaattcatcaccctaatgatgcagctgaacagataaaactctaccagaacccgaacacttacgtgtcagtgggaacatcaacactgaatcagaggtcaatcccagaaatagccaccagacccaaggtgaacggacagagtggaaggatggagtttttttggacaatactaaggccgaacgactcaatcacctttgagagtactgggaactttatagctcccgaatatgcatacaagattgttaaaaaaggagactcaacaatcatgaaaagtgaactgagttatggtaactgtgataccaaatgtcagaccccagtgggtgctataaattccagtatgcctttccacaatgtccatccctttaccattggggagtgccccaagtatgtcaaattgaaaaaactggtccttgcaacagggctaagaaacgtaccccaaagagaaaaaagaggcctatttggagcaatagccggattcatagaaggaggatggcaaggaatggtggatggatggtatggataccatcatagcaatgagcagggtagtggatatgctgcagacaaagaatctacacagaaagcagtcgatgggatcactaataaagtcaactcaatcattgacaaaatgaacactcaattcgaagccgttggaaaagaattcaacaacctagaaaggagaatagaaaatttgaataagaaaatggaagatggctttatagatgtatggacttacaatgcagaacttctagtgctcatggaaaacgaaagaactctggatctccatgattcaaatgttaagaacttatacgataaggtccgacaccagctgagagacaatgcaaaagaattaggcaatgggtgctttgaattctatcacaggtgtgacgataaatgcatggaaagtgtgagaaatggaacgtatgactatccacaatactcagaagaatcaagactgaaaagggaggaaatagacggagtcaaattagaatcaatgggggcttatcagatactttcaatctattcaacagtagcgagttccctggcactggcaatcatggtagctggtctatctttttggatgtgttccaatggatcattgcagtgcagaatttgcatctaaaattgtgagttcagattgtagttaaaaacacccttgtttctact

SEQ ID NO: 3 - DNA, 43 bp, artificial sequence
  Forward primer for amplifying gp64 signal sequence.
atatctcgagatgctactagtaaatcagtcacaccaaggcttc

SEQ ID NO: 4 - DNA, 18 bp, artificial sequence
  Reverse primer for amplifying gp64 signal sequence.
gtggtggtggtggtggtg

SEQ ID NO: 5 - DNA, 63 bp, artificial sequence
  Forward primer for amplifying mature domain of hemagglutinin.
gcgcaccaccaccaccaccacggtatggctgaccgaatctgcattggttatcatgcaaacaat

SEQ ID NO: 6 - DNA, 61 bp, artificial sequence
  Reverse primer for amplifying mature domain of hemagglutinin.
atatggtaccttaatattgtctattacggtttctggaacacatccaaaaagatagaccagc

SEQ ID NO: 7 - DNA, 20 bp, artificial sequence
  Reverse primer for amplifying mature domain and C-terminal domain of hemagglutinin.
ttcagttagcctcccccatc